(19) United States
(12) Patent Application Publication
(10) Pub. No.: US 2021/0353772 A1
(43) Pub. Date: Nov. 18, 2021

(54) METHOD OF MAKING A BIOCOMPATIBLE MICRO-SWIMMER AND METHOD OF USING SUCH A MICRO-SWIMMER

(71) Applicants: MAX-PLANCK-GESELLSCHAFT ZUR FÖRDERUNG DER WISSENSCHAFTEN E.V., München (DE); Koç University, Sariyer/lstanbul (TR)

(72) Inventors: Metin Sitti, Stuttgart (DE); Hakan Ceylan, Stuttgart (DE); Oncay Yasa, Stuttgart (DE); Immihan Ceren Yasa, Stuttgart (DE); Seda Kizilel, Sariyer/lstanbul (TR); Ugur Bozüyük, Sariyer/lstanbul (TR)

(21) Appl. No.: 17/274,215

(22) PCT Filed: Sep. 10, 2018
(86) PCT No.: PCT/EP2018/074311

§ 371 Date: Mar. 8, 2021

(51) Int. Cl. A61K 47/69; A61K 41/00; A61K 47/61; A61K 9/50; B33Y 10/00; B33Y 80/00; B29C 64/135; B33Y 70/10

(52) U.S. Cl. CPC A61K 47/6957; A61K 41/0042; A61K 47/61; A61K 9/5094; B29K 2105/0035; B33Y 80/00; B29C 64/135; B33Y 70/10; B29K 2995/0056; B33Y 10/00

(57) ABSTRACT

The present invention relates to a method of making a biocompatible micro-swimmer, the method comprising the steps of: providing a photo cross-linkable biopolymer solution; adding magnetic particles and a photo initiator to the photo cross-linkable biopolymer solution to form a 3D-printable solution; applying a laser with a variable focus directed at the 3D-printable solution; varying the focus of the laser through the 3D-printable solution to form the biocompatible micro-swimmer with a predefined shape; and applying a chemical linker to the biocompatible micro-swimmer having the pre-defined shape. The invention further relates to such a micro-swimmer and to a method of using such a micro-swimmer.

The present invention relates to a method of making a biocompatible micro-swimmer. The invention further relates to such a micro-swimmer and to a method of using such a micro-swimmer.

Microscopic swimmers powered by external magnetic fields possess significant potential in medical applications due to their wireless actuation, active locomotion and precise localization capabilities. Their small size and untethered control could allow deep tissue penetration, and thus could revolutionize minimally invasive surgeries and therapies. So far, synthetic magnetic micro-swimmers which are actuated using an external power source have been used in different platforms for targeted cargo delivery, object/cell manipulation and tissue engineering applications. Particularly, helical magnetic micro-swimmers have recently gained interest due to the efficiency of magnetic torque over magnetic gradient pulling for microscale actuation.

Helical micro-swimmers, operated in low Reynolds number regime with an external rotating magnetic field, were previously designed in millimeter scale using a small magnet incorporated at the head of a spirally-bent cupper wire. Then, different fabrication techniques, including self-scrolling and glancing angle deposition, were utilized to fabricate helical magnetic swimmers at micron scale. Afterward, the advancements in two-photon direct laser writing (TDLW) technique realized three-dimensional (3D) fabrication of more complex polymeric microstructures, eased their local 3D patterning using versatile chemical moieties and provided the possibility to embed biocompatible superparamagnetic iron oxide nanoparticles (SPIONs) into the micro-swimmers. Up until now different photosensitive materials have been used with TDLW technique to fabricate helical micro-swimmers. Initially, helical micro-swimmers functionalized with drug-loaded liposomes were utilized to perform single cell drug delivery in vitro.

Despite the recent developments in the field, helical magnetic micro-swimmers still need to be strengthened to have physiologically-relevant biodegradation and controlled local cargo release capabilities, which are essential for their potential medical applications.

Biodegradation of administered micro-swimmers inside the body in a known period of time by forming non-toxic degradation products is a critical aspect of medical applications. Recently, degradation of helical micro-swimmers, composed of various ratios of PEG-DA/PE-TA and SPIONs, through sodium hydroxide based hydrolysis reaction was demonstrated. However, usage of 1 M NaOH solution for the degradation of the micro-swimmers could be problematic, and hence integration of new natural, physiologically-relevant degradation mechanisms to the micro-swimmers is indispensable for future medical applications.

In addition, a controlled release of concentrated therapeutics at disease sites by active micro-swimmers could increase the overall treatment efficiency. Helical micro-swimmers overcome active delivery issues of therapeutics to site of action using rotating magnetic fields.

However, controlled release of the therapeutics is still an issue, which should be addressed in micro-swimmer-based drug delivery systems. Remotely-triggered systems have always been attractive for facilitating release of therapeutics to desired sites at desired times.

For this reason it is an object of the present invention to make available a biodegradable micro-swimmer that is not and that does not form any toxic degradation products so that the micro-swimmers can readily be used in a wide range of medical applications. It is a further object of the invention to make available a micro-swimmer by means of which a controlled active release of the cargo material is possible to ensure an on-demand, precise and effective delivery of the cargo material. It is yet a further object of the present invention to make available a micro-swimmer that can be guided to a desired target region without causing excessive harm to the tissue surrounding the target region.

This object is satisfied by method of making a biocompatible micro-swimmer in accordance with claim 1. Further benefits and advantageous embodiments of the invention will become apparent from the dependent claims, from the description and from the accompanying drawings.

Such a method may comprise the steps of:
providing a photo cross-linkable biopolymer solution;
adding magnetic particles and a photo initiator to the photo cross-linkable biopolymer solution to form a 3D-printable solution;
applying a laser with a variable focus directed at the 3D-printable solution;
varying the focus of the laser through the 3D-printable solution to form the biocompatible micro-swimmer with a predefined shape; and
applying a chemical linker to the biocompatible micro-swimmer having the pre-defined shape.

By forming the micro-swimmer with a photo cross-linkable biopolymer solution the micro-swimmers can be made in a fast and efficient manner using 3D printing technologies.

Such a 3D printing technology permits the formation of micro-swimmers, with a micro-swimmer being defined as a component having at least one dimension of the micro-swimmer is selected in the range of 0.0001 to 1 mm.

Moreover, on use of a biopolymer solution to form the micro-swimmers, the micro-swimmers can be formed such that they themselves nor their degradation products form a toxic response inside a living environment. This makes available the possibility of using such magnetic micro-swimmers in parts of the body that are not directly connected to the gastro-intestinal tract.

Furthermore, the provision of a chemical linker at the micro-swimmer means that different chemical substances and other materials can be attached to the micro-swimmer in a simple manner thereby making available a micro-swimmer that is capable of transporting cargo material to a desired target region.

Through use of magnetic particles present within the micro-swimmer the cargo material can be delivered in a controlled and targeted manner to a desired target region thereby ensuring an on-demand, precise and effective delivery of the cargo material to the target region.

The chemical linker may form a link between the biocompatible micro-swimmer and a cargo that is attachable to and transportable by the micro-swimmer. In this way the cargo may be chemically bonded to the micro-swimmer.

The method may further comprise the step of attaching a cargo at the biocompatible micro-swimmer via the chemical linker. The cargo material can thus be chemically bonded to the micro-swimmer and thereby be present e.g. on the surface of the micro-swimmer to allow an efficient release of the cargo material at the desired target region.

The chemical linker is preferably selected such that the link between the micro-swimmer and the cargo can be released on the presence of a stimulus. In this way a micro-swimmer is formed by means of which a controlled active release of the cargo material is possible to ensure an on-demand, precise and effective delivery of the cargo material.

In this connection it should be noted that the cargo may be selected from the group of members consisting of enzymes, molecules, drugs, proteins, genetic materials, nanoparticles, radioactive seeds for therapeutic or diagnostic purposes and combinations of the foregoing.

The chemical linker may be a photo cleavable linker, preferably an NHS and Alkyne modified o-nitronezyl derivative. Through the use of a photo cleavable linker a cargo material can be released from the micro-swimmer by means of e.g. laser light, for example infrared or ultraviolet laser light.

In this connection it should be noted that the chemical linker may be an enzymatically cleavable linker, for example, one of the matrix metalloproteinase recognition peptide sequences. In this way the cargo material can be released from the micro-swimmer in the presence of specific enzymes, e.g. the enzymes present in cancerous tissue, i.e. the stimulus is provided by a certain level of specific enzymes.

It should further be noted that the chemical linker may be a thermally cleavable linker that is configured to release the cargo material under the influence of heat, i.e. the stimulus is provided by the application of a temperature within a certain range.

The photo-crosslinkable biopolymer solution may be a solution comprising bioactive, biodegradable and biocompatible polymers, for example, chitosan, gelatine, alginate, polypeptides, nucleic acids, polysaccharides and combinations of the foregoing, preferably chitosan. In this way a micro-swimmer is made available that can be formed from readily abundant and comparatively inexpensive materials that do not form toxic reactions within the host into which the micro-swimmers may be introduced.

In this connection it should be noted that the term biodegradable means that the biocompatible micro-swimmer degrades over time within a living organism by enzymatic activity and without causing damage to the surrounding tissue. This is not the case for micro-swimmers known from the prior art that are degraded through sodium hydroxide based hydrolysis reactions. Such reactions form toxic byproducts and hence would cause serious harm to tissues in the human or animal body.

It should further be noted that the magnetic particles are a colloidal particles that are homogeneously suspended in the photo cross-linkable biopolymer solution prior to forming the micro-swimmers on the application of the laser.

The magnetic particles have a size selected in the range of 5 nm to 200 nm, in particular 5 to 100 nm, and preferably 40 to 60 nm. In this way the 3D-printable solution can be made available in which a homogenous dispersion of magnetic particles is made possible. Magnetic particles or agglomerations of magnetic particles greater than 200 nm in size present within a micro-swimmer and subjected to changing magnetic field strengths can accidentally cause the micro-swimmer to deviate from the desired path and hence reduce the steering capability of the micro-swimmers. Moreover, magnetic particles or agglomerations of magnetic particles greater than 200 nm in size become incompatible with TDLW printing technology. Therefore, the structural fidelity goes lower.

The magnetic particles may be selected from the group of members consisting of iron oxide particles, iron platinum particles, neodymium iron boron particles, aluminum nickel cobalt particles, iron particles, cobalt particles, samarium cobalt particles. Preferably iron oxide particles are used as this material is known to be biocompatible and non-toxic within the host.

The photo initiator is a molecule that upon two photon absorption splits into half and generates radicals that initiate the photo-crosslinking, with the photo initiator, for example, being LAP. Through the use of a photo initiator that reacts using a two-photon absorption it is possible to form 3D micro-swimmers with sizes of length in the range of, for example, 1 to 1000 μm and width, for example, in the range of 0.1 to 100 μm.

In this connection it should be noted that the photo initiator is ideally water soluble. In order to be able to be used in a 3D printer the photo initiator has to be able to absorb photons at the wavelength of the 3D printer so as to generate the radicals and consequently form the micro-swimmer of the desired shape. For this purpose it is ideal if the photo initiator has a two-photon cross-section that allows radical generation with two photon absorption.

The cargo is preferably releasable from the micro-swimmer on the application of a stimulus, for example the application of light, or in the vicinity of predefined amount of specific enzymes due to a pathological condition within the host, e.g. in present in the vicinity of specific cancerous cells.

The method may further comprise the step of applying a magnetic field whose magnetic field strength is selected in the range of 5-30 mT is selected in order to align the magnetic particles within the 3D-printable solution during the step of applying the laser. In this way a magnetic orientation of the micro-swimmer can be predefined.

The micro-swimmer may have a shape that is configured to be asymmetrically moved in dependence on time in the presence of a rotating magnetic field, such as a helical or double helical shaped structure. By forming the micro-swimmer in such a way enables the micro-swimmer to be steered and moved more accurately.

The micro-swimmer may have an elongate shape, with a ratio of length to width being selected in the range of 2:1 to 10:1. Such shapes can be moved in an advantageous manner using a rotating magnetic field and enable desired amounts of cargo to be transported with a micro-swimmer.

According to a further aspect the present invention further relates to a biocompatible micro-swimmer, in particular made using a method as discussed herein, the micro-swimmer comprising a body portion formed of a 3D printable solution including a photo cross-linkable biopolymer solution, magnetic particles and a photo initiator; wherein the body portion of the micro-swimmer has a shape that is configured to be asymmetrically moved in dependence on time in the presence of a rotating magnetic field, such as a helical or double helical shaped structure; and wherein the body portion is coated with a chemical linker.

The micro-swimmer can ideally be further developed in accordance with the method of making described in the foregoing, thereby the micro-swimmer can have the resultant advantages described in connection with the method of making.

The micro-swimmer can hence be produced at one site and then shipped to a further site where it can then be loaded with a cargo. For example, if the cargo material is a radioactive imaging agent it is beneficial if the micro-swimmer is not yet loaded on shipping to e.g. the radiology lab with the cargo-material, but only shortly prior to its use to prevent the radioactive material from decaying and hence becoming inactive.

The provision of a biodegradable micro-swimmer makes it possible to eliminate previously required retrieval steps, since the micro-swimmer will simply decompose in the host and during this decomposition does not form any toxic reactions that could lead to any harm.

The body portion of the micro-swimmer may have an elongate shape, with a ratio of length to width being selected in the range of 2:1 to 10:1. In this connection it should be noted that at least one dimension of the micro-swimmer may be selected in the range of 0.0001 to 1 mm.

Advantageously the micro-swimmer may be configured to be moved with a Reynold's number of less than 0.1. This ensures that the micro-swimmer can be moved within the host in a controlled manner.

The micro-swimmer may be magnetised in a direction perpendicular to its major axis, i.e. perpendicular to its elongate extent. This enables a magnetic orientation of the micro-swimmer to be predefined.

The micro-swimmer may be configured to degrade such that within a period of 210 hours in a solution having a Lysozyme concentration of 1.5 μg/ml a length of the micro-swimmer degrades to a length of at most 70%, preferably at most 65%, of the initial length and a diameter of the micro-swimmer degrades to a diameter of at most 50%, preferably of at most 45%, of the initial diameter of the micro-swimmer. This is a further indication of the biocompatibility of the micro-swimmer.

According to a further aspect the present invention relates to a method of using one micro-swimmer loaded with cargo material as discussed in the foregoing. The method comprising the steps of:
providing the micro-swimmer in a region associated with the desired target region;
directing the micro-swimmer with a time variable magnetic field to the desired target region;
stimulating the micro-swimmer in the desired target region to release the cargo.

In this way a concentration of therapeutics, i.e. of cargo material, at the site of action can be controlled and increased in comparison to prior art systems. Moreover, the overall injected dose can be decreased using remotely-triggered systems in comparison to the prior art. By providing e.g. a light stimulus, a light-triggered release is made available which is especially practical. Other trigger or stimulating mechanisms may include pH, temperature, ultrasound and magnetic field, due to their high spatiotemporal accuracies.

Using ultraviolet (UV) light-triggered release systems, the poor tissue penetration depth of the UV light restricts the number of potential medical applications to certain locations inside the human or animal body to those regions close to the skin. However, optical upconversion processes, in which low-energy photons (e.g., near-infrared light that has more penetration depth) may be transformed to high-energy photons within the body (e.g., UV light). Such systems may be utilized to enable the stimulation of the micro-swimmers in regions of the human or animal body that cannot be penetrated using UV light thereby increasing the number of possible medical applications in different parts of the body.

The step of directing may comprise the application of a rotating field strength in the range of 5 mT to 50 mT with a frequency selected in the range of 1 Hz to 50 Hz. The use of magnetic fields in and around the human and/or animal body can be carried out in a beneficial manner without any known side effects.

The step of stimulating the micro-swimmer in the desired target region to release the cargo is carried out by applying a light stimulus at the target region. The application of a light stimulus has been found to yield an efficient trigger mechanism for the targeted release of the cargo material at the desired target region.

The step of directing the micro-swimmer may be conducted in conjunction with image mapping, e.g. using an MRI, in order to track a path of the micro-swimmer to the desired target region. This advantageously enables a feedback of the current position of the micro-swimmer and also permits a more precise targeted stimulation of the release of the cargo.

The invention will be described in the following by way of embodiments in detail with reference to the Drawing, in which is shown:

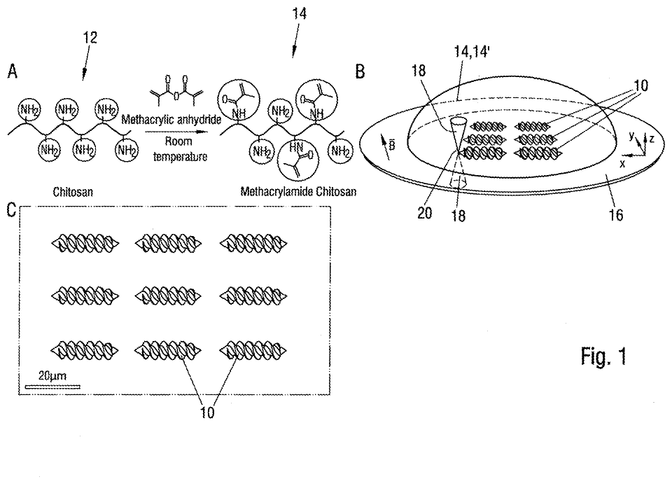

FIGS. 1 A to C an overview of the synthesis and fabrication processes and of the resultant micro-swimmers, with A) detailing the synthesis of the photo cross-linkable solution, B) illustrating the 3D printing of micro-swimmers using two-photon direct laser writing technique, C) illustrating an optical microscopy image of a 3D printed 3×3 array of the micro-swimmers;

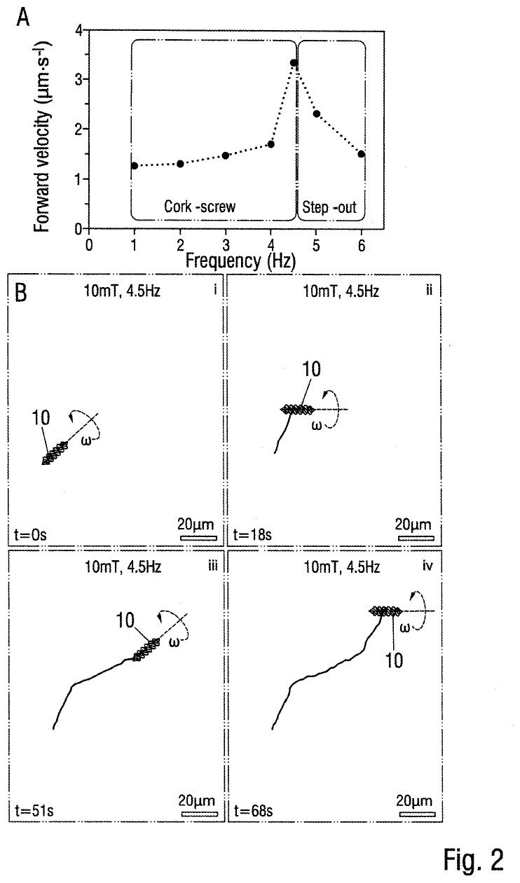

FIGS. 2A &B actuation and steering capabilities of the micro-swimmers using a rotating magnetic field, with FIG. 2A) illustrating a forward velocity of the micro-swimmers as a function of magnetic excitation frequency, and with FIG. 2B) illustrating controlled swimming trajectory snapshots (dashed lines) of the micro-swimmers on the application of a 10 mT rotating magnetic field at 4.5 Hz illustrated with the dotted line at w;

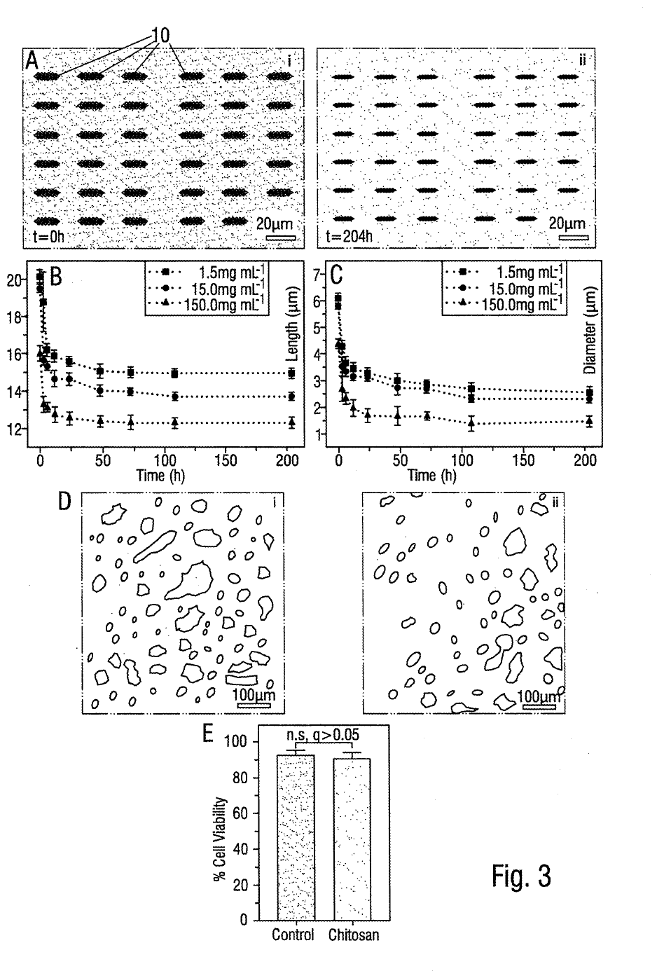

FIGS. 3A to E enzymatic degradation of the micro-swimmers using lysozyme, with FIG. 3A) illustrating optical microscopy images of the micro-swimmers treated with 15 μg·mL⁻¹ lysozyme, with FIG. 3Ai at time t=0 h and FIG. 3Aii at time t=204 h, which reveal a surface corrosion-based degradation mechanism; FIG. 3B) illustrating changes in length of the micro-swimmers in time with different lysozyme concentrations, FIG. 3C) illustrating changes in diameter of the micro-swimmers in time with different lysozyme concentrations; FIG. 3D) illustrating dead staining of SKBR3 breast cancer cells with FIG. 3Di showing non-treated and FIG. 3Dii. illustrating those cells treated with the degradation products of the micro-swimmers for a duration of 1 day, with open dots representing live cells and solid dots representing dead cells, FIG. 3E) illustrating a quantification of viability of SKBR3 breast cancer cells treated with the degradation products;

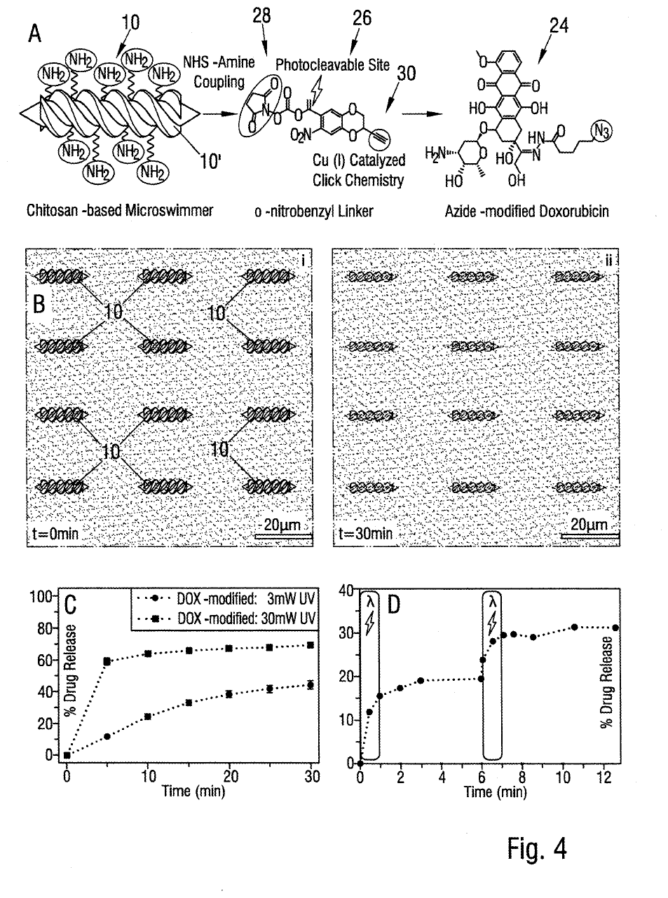

FIGS. 4A to D the process of a light-triggered drug release from the micro-swimmers, with FIG. 4A showing a schematic reaction pathway to obtain DOX-modified micro-swimmers, with amino groups on the micro-swimmers reacting with NHS group of o-nitrobenzyl photocleavable chemical linker molecules, and the following azide-modified DOX reaction with alkyne ends of the micro-swimmers, with FIG. 4B illustrating the DOX release from the micro-swimmers exposed to 30 mW light intensity i) at a time t=0 mins and FIG. 4Bii at a time t=30 min and a decrease in the fluorescence intensity indicating the cleavage of DOX from the micro-swimmers and its release; FIG. 4C) illustrating the cumulative DOX release from the micro-swimmers for 3 mW (round dots) and 30 mW (square dots) light intensity; and FIG. 4D) showing smart dosing of DOX from the micro-swimmers, with the solid bars at t=0 to 1 min and t=6 to 7 min illustrating the application of a laser with 365 nm wavelength and that approximately 15% of DOX was released per minute from the micro-swimmers;

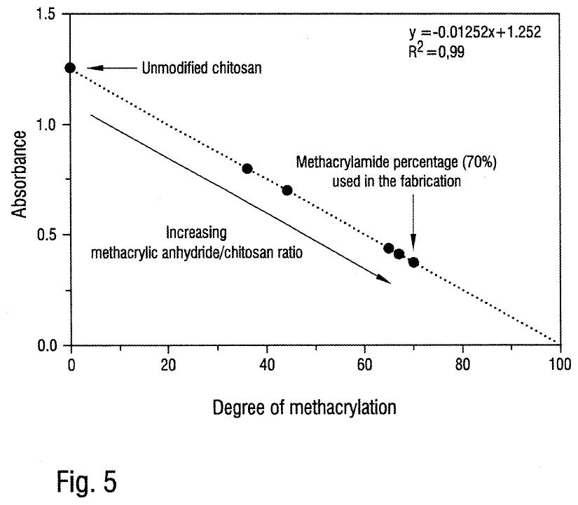

FIG. 5 determination of degree of methacrylation using 2,4,6-trinitrobenzene sulfonic acid (TNBS) assay;

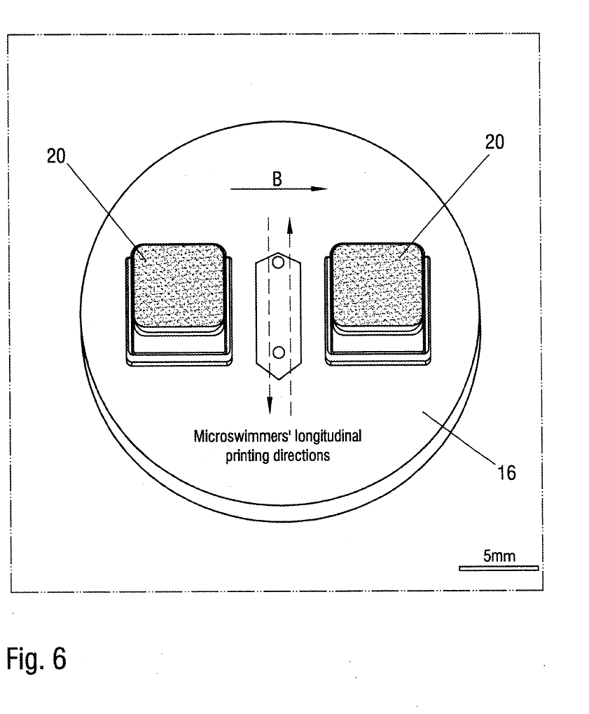

FIG. 6 a photograph of a microchannel setup utilized for two-photon-based 3D-printing of the micro-swimmers;

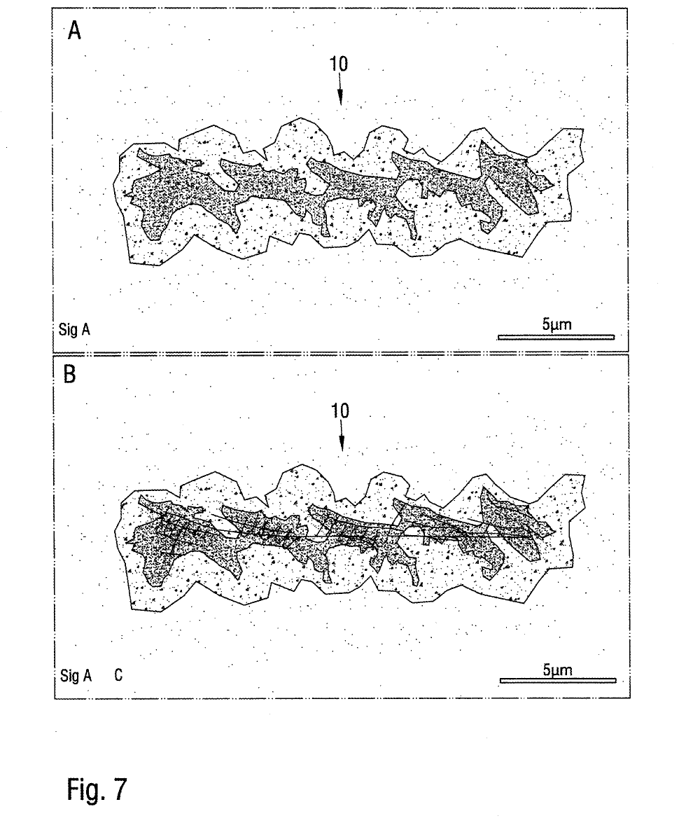

FIGS. 7A & B energy-dispersive x-ray spectroscopy elemental mappings of the micro-swimmers performed at 15 keV for 10 min, with FIG. 7B showing the presence of carbon atoms within the micro-swimmers;

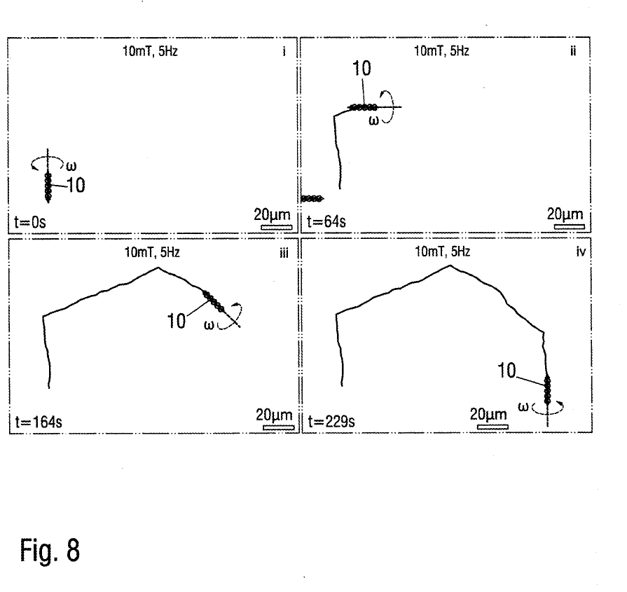

FIG. 8i to iv controlled swimming trajectory snapshots (dotted lines) similar to FIG. 2B of the micro-swimmers swum at 5 Hz under a 10 mT rotating magnetic field illustrated at w;

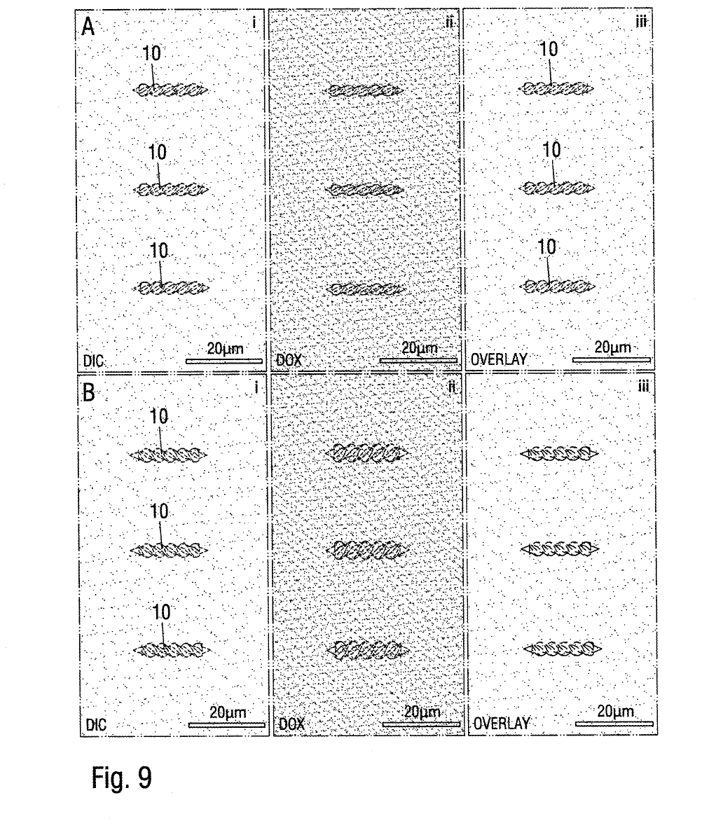

FIGS. 9A & B chemical integration of the drug molecules to the micro-swimmers. A) Micro-swimmers without the o-nitrobenzyl linker modification were directly treated with the drug molecules (azide-DOX). B) Micro-swimmers with the o-nitrobenzyl linker modification were treated with the drug molecules (azide-DOX). Images were captured with the same fluorescence intensity and exposure time, and represent the controlled integration of azide-DOX onto the micro-swimmers;

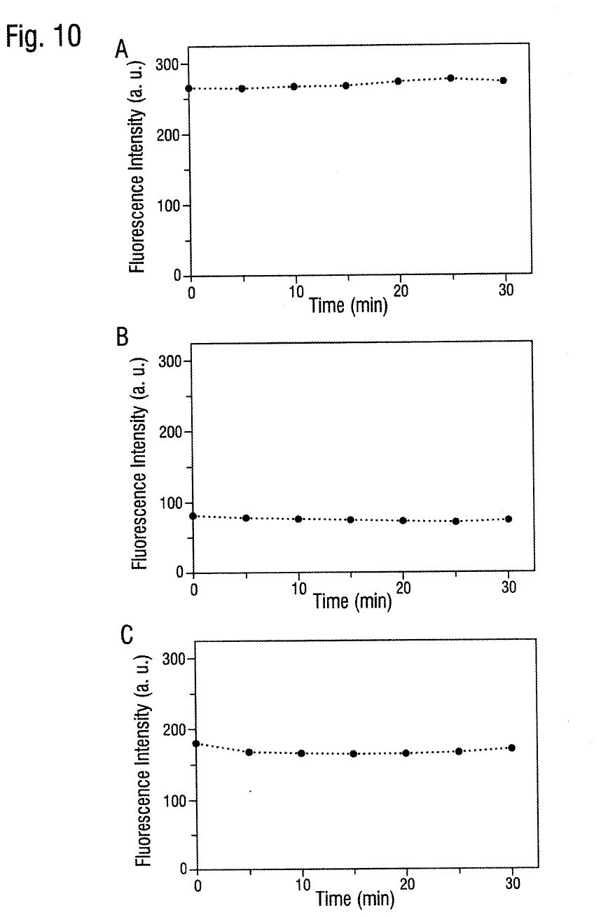

FIGS. 10A to C photobleaching tests for the drug molecules prior to controlled release experiments, with FIG. 10A) illustrating 470 nm wavelength light excitation for 30 min to negative group (micro-swimmers without the o-nitrobenzyl linker modification), FIG. 10B) illustrating 3 mW 365 nm wavelength light exposure for 30 min to negative group, and FIG. 100) illustrating 30 mW 365 nm wavelength light exposure for 30 min to negative group;

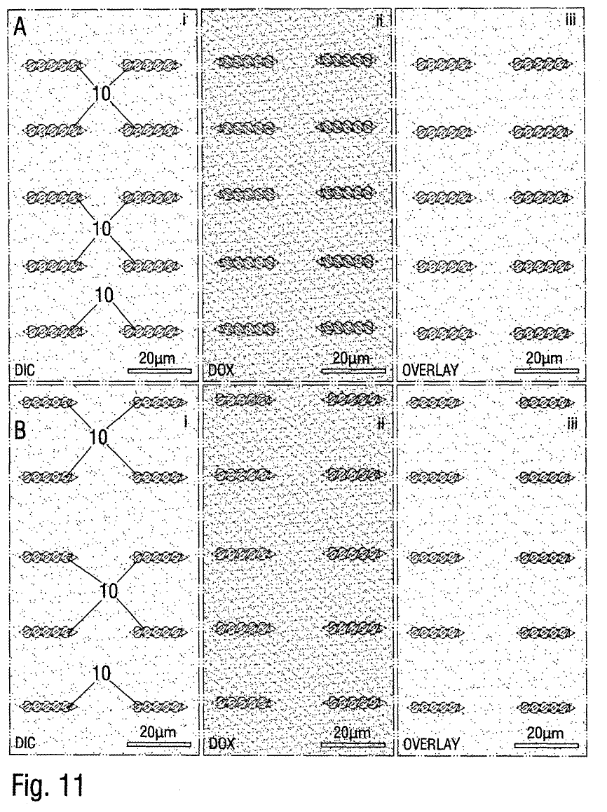

FIGS. 11A & B images showing controlled and localized drug release from the micro-swimmers, with FIG. 11A) illustrating a 365 nm wavelength light exposure focused onto the micro-swimmers (in the left column), controlled release of drug molecules upon light exposure (middle column), while the remaining group (right column) still had the integrated drug molecules; and FIG. 11B) illustrating a precise and controlled drug release from half of the micro-swimmers body.

Features which have the same or a similar function will be described in the following using the same reference numeral. It is also understood that the description given with respect to reference numerals used in one embodiment also applies to the same reference numerals in connection with other embodiments unless something is stated to the contrary.

FIGS. 1 A to C show an overview of the processes of making a biocompatible micro-swimmer 10 and the resultant micro-swimmers 10. The method comprises the steps of providing a photo cross-linkable biopolymer solution 12 in a container and adding magnetic particles and a photo initiator (both not shown) to the photo cross-linkable biopolymer solution to form a 3D-printable solution 14.

The 3D printable solution 14 in the example of FIG. 1A is formed by preparing in 8% (v/v) acetic acid containing ddH₂O, and composed of 30 mg·mL⁻¹ ChMA, 20 mg·mL⁻¹ phenyl-2,4,6-trimethylbenzoylphosphinate (LAP) photoinitiator and 5 mg·mL⁻¹ PEG/Amine-functionalized 50 nm biocompatible superparamagnetic iron oxide nanoparticles (SPIONs). This solution is subsequently stirred and sonicated for a time of 15 hours.

Chitosan is a linear and cationic polymer which is obtained from chitin, the second most abundant natural polymer in the world. Its inherent properties, such as biocompatibility, biodegradability, bioadhesivity, and antimicrobial, antitumor and antioxidant activities, make chitosan an ideal polymer for medical applications.

In this connection it should be noted that the photo-crosslinkable biopolymer solution 12 is a solution comprising bioactive, biodegradable and biocompatible polymers, for example, chitosan, gelatine, alginate, polypeptides, nucleic acids, polysaccharides and combinations of the foregoing and as indicated in the foregoing the preferred choice is chitosan.

Polymers without photosensitive characteristics like chitosan can be chemically modified while their polysaccharide backbones remain unchanged. For this reason a photosensitive form of chitosan, methacrylamide chitosan (ChMA), was initially prepared. This was performed by reacting amino groups of the polymer with methacrylic anhydride. The amino groups of the chitosan transformed into photosensitive methacrylamide groups according to the methacrylic anhydride/chitosan ratio at constant reaction time (FIG. 1A).

The newly formed polymer chains then possess the capability of being crosslinked with one another, in the presence of a photo initiator, and UV light with a wavelength of around 350 nm wavelength.

In this connection it should be noted that the photo initiator is a molecule that upon two-photon absorption splits into half and generates radicals that initiates the photo-crosslinking, with the photo initiator, for example, being the aforementioned LAP. The photo-crosslinking capability is required to form solid micro-swimmers 10 from the 3D printable solution 14.

After the synthesis of the 3D polymer solution 14, a methacrylation degree of ChMA macromolecules was determined using 2,4,6-trinitrobenzene sulfonic acid (TNBS) assay. The results of this test are discussed in connection with FIG. 5. TNBS is a photospectroscopic reagent used to determine free amino groups. Methacrylic anhydride/chitosan ratio was changed at fixed reaction time and the assay demonstrated that amino groups were consumed with increasing methacrylic anhydride/chitosan ratio.

In order to facilitate the printing time methacrylamide chitosan macromolecules with 70% methacrylation degree were selected for 3D printing. For this ChMA macromolecules with a backbone composed of approximately 70% photosensitive methacrylamide groups were selected for the fabrication procedure.

As indicated in FIG. 1B a liquid drop 14′ of the 3D printing solution 14 is placed on a substrate 16 in the form of a petri dish. Six micro-swimmers 10 are indicated on the petri dish. Also indicated are two laser beams 18 focused on a focal point 20.

In order to form the micro-swimmers 10, the lasers 18 used have a variable focus, this means that the position of the focal point 20 of the laser 18 can be changed in a pre-determinable way by varying e.g. a focal length of the respective laser 18 or the position of the laser 18 relative to the substrate 16 to move the focal point 20. This variation of the position of the focal point 20 can take place in all three spatial dimensions x, y and z as indicated by the origin in FIG. 1B. On application of the lasers 18 and by varying the focus of the lasers 18 through the 3D-printable solution 14 the biocompatible micro-swimmers 10 can be formed with a predefined shape.

The chitosan-based microswimmers 10 shown in FIGS. 1B and 1C were fabricated in double helices geometry using two-photon direct laser writing (TDLW) technique. In this way photopolymerization of the prepolymer of the 3D printable solution 14 to form the predefined structures was performed in a close channel. Generally speaking the micro-swimmers 10 have a shape that is configured to be asymmetrically moved in dependence on time in the presence of a rotating magnetic field, such as a helical or the double helical shaped structure.

It should further be noted that the magnetic particles added to form the 3D-printable solution 14 have a size selected in the range of 5 nm to 200 nm, in particular 5 to 200 nm, preferably 40 to 60 nm. The magnetic particles are preferably SPIONs due to their biocompatibility, but other biocompatible magnetic particles may be used.

The reason for this is that the usage of SPIONs in the design of the micro-swimmer 10 has two main advantages: (1) SPIONs are considered to be biocompatible and to have no severe side effects in vivo, and (2) the SPIONs dramatically increase the availability of drug and cargo release sites compared to cobalt- or nickel-based surface coatings.

The micro-swimmers 10 may have an elongate shape, with a ratio of length to width being selected in the range of 2:1 to 10:1 and with at least one dimension of the micro-swimmer being able to be selected in the range of 0.0001 to 1 mm.

In order to be able to move the micro-swimmers 10 in a magnetic field, the micro-swimmers have to be magnetized. For this purpose, see also FIG. 6, two permanent magnets 22 are placed on the petri dish 16 at either side of the region where the micro-swimmers 10 are formed, so that a magnetic field B is applied in the direction of the arrow. The magnetic field B strength is selected in order to align the magnetic particles within the 3D-printable solution 14 during the step of applying the laser 18 on forming the micro-swimmers 10.

By arranging the permanent magnets 22 in a pre-defined manner the magnetic orientation of the SPIONs is aligned. The SPIONs present in the micro-swimmer 10 having an aligned magnetic orientation can subsequently be controlled and steered, so that the micro-swimmers can be moved in 3D aqueous environments using rotating magnetic fields.

The average printing rate was around 10 seconds for an individual micro-swimmer 10. Energy-dispersive x-ray spectroscopy (EDS) elemental mapping carried out on the formed micro-swimmers confirmed a homogenous dispersion of iron atoms in the micro-swimmers 10.

As further indicated in FIG. 1C the micro-swimmers 10 have a 6 μm diameter and a 20 μm length and are composed of double helices to operate in low Reynolds number regime with a low-amplitude rotating magnetic field. The micro-swimmers 10 shown in FIG. 1C are capable of being actuated and controlled in an aqueous environment with an average speed of 3.34±0.71 μm·s⁻¹ using a 10 mT and 4.5 Hz rotating magnetic field. In this connection it should be noted that the low Reynolds number regime is a regime having a Reynold's number of less than 0.1.

FIGS. 2A &B shows that the micro-swimmers 10 are capable of being actuated and steered using a rotating magnetic field. FIG. 2A shows the forward velocity of the micro-swimmers 10 as a function of magnetic excitation frequency. As demonstrated, the micro-swimmers 10, having the sizes illustrated in FIG. 1C, have a step-out frequency of 4.5 Hz.

In order to do this, the micro-swimmers 10 were actuated and steered using a five-coiled electromagnetic setup (not shown). The five-coiled electromagnetic setup can be mounted on an inverted optical microscope (not shown) in order to track the motion of the micro-swimmers 10. The five coiled magnetic setup can be controlled in a manner known per se to generate and control the desired rotational magnetic field, e.g. in the range of 2 to 50 mT with a frequency selected in the range of 1 Hz to 50 Hz with a uniformity above 95% across a 2 cm×2 cm×2 cm volume.

This means that the gradients and the orientation of the magnetic field can be varied in order to direct the micro-swimmers 10 in the desired direction. The precise field strength and frequency of the magnetic field may generally be selected in dependence on the size of the micro-swimmer and the amount of SPIONs consequently present therein.

The results shown in FIG. 2A were recorded using a 10 mT rotating magnetic field. Initially, a step-out frequency of the micro-swimmers was investigated by gradually increasing the frequency of the applied rotating magnetic field from 1 Hz to 6 Hz with 0.5 Hz steps. It was demonstrated that the fabricated micro-swimmers were actuated and steered optimally at 4.5 Hz under a 10 mT rotating magnetic field. The average forward velocity of the micro-swimmers at the optimum actuation frequency of 4.5 Hz was measured to be 3.34±0.71 μm-sec′.

By placing the five-coiled electromagnetic setup on an inverted optical microscope it is possible to steer the micro-swimmers 10 in different paths and to record their progression on these paths by the microscope in order to demonstrate and record images of the controllability of the microsystem. It was shown that it is possible to steer the micro-swimmers at both 4.5 Hz and 5 Hz under a 10 mT rotating magnetic field (FIG. 2Bi to iv and FIG. 8), in this connection it should be noted that FIGS. 8i to 8iv show images similar to those of FIG. 2Bi to iv, the difference being the frequency of the applied magnetic field.

FIGS. 3A to E show the enzymatic degradation of the micro-swimmers 10 using lysozyme. As mentioned in the foregoing biodegradable materials have gained increasing attention in medicine, since they are able to naturally break down and disappear from the body after performing their functions. Chitosan, as a biodegradable material, is primarily degraded by lysozyme enzyme, which is present in various tissues and body fluids with a range of approximately 1-15 μg·mL⁻¹ concentrations. This effect is due to the lysozyme enzyme cutting off the glycosidic bonds between monomers in the polymer backbone and the resulting small chains are removed naturally.

FIGS. 3Ai and 3Aii illustrate optical microscopy images of 3D printed arrays of the micro-swimmers 10, with FIG. 3Ai illustrating the start of the biodegradation experiment conducted on the micro-swimmers 10 and FIG. 3Aii showing the occurred surface erosion, in which water and enzymes could not penetrate inside the crosslinked structures; and thus, started to degrade initially the exterior surface of the micro-swimmers 10. It was shown that the helices and sharp edges of the micro-swimmers 10 were degraded first by the lysozyme enzyme. The micro-swimmers 10 were partially degraded after 204 hours as indicated in FIG. 3Aii which shows the degradation in a 15 μg·mL⁻¹ lysozyme concentration.

In order to test the degradation of the micro-swimmers 10, three different lysozyme enzyme concentrations (1.5 illustrated by the triangular points in FIGS. 3B and C, 15 illustrated by the round points in FIGS. 3B and C and 150 μg·mL⁻¹ illustrated by the triangular points in FIGS. 3B and C) were chosen for the biodegradation of the micro-swimmers 10, where 150 μg·mL⁻¹ represented an unrealistically high condition.

FIG. 3B shows the changes in length of the micro-swimmers 10 in time for the different lysozyme concentrations and FIG. 3C shows the changes in diameter of the micro-swimmers in time for the different lysozyme concentrations. For all concentrations it was found that a length of the micro-swimmer 10 degrades to a length of at most 70% of the initial length and a diameter of the micro-swimmer 10 degrades to a diameter of at most 50% of the initial diameter of the micro-swimmer 10 within a period of time of 210 hours.

As expected, the unrealistically high lysozyme enzyme concentration group (150 μg·mL⁻¹) result in the fastest degradation with the smallest diameter and length micro-swimmers 10 remaining. Whereas the 1.5 μg·mL⁻¹ lysozyme concentration group had the largest micro-swimmers 10 remaining after 204 hours (FIGS. 3B and 3C). There was rapid diameter and length changes in all groups since helices and edges were degraded first due to the surface erosion. After biodegradation of the helices and the edges, which had smaller volume compared to whole body of the micro-swimmers 10, the rate of diameter and length changes dramatically decreased as expected.

This did not necessarily mean decrease in the biodegradation rate, because lysozyme enzyme then tried to degrade cylindrical micro-swimmer body 10′ which had lower surface area to volume ratio compared to helices. Because of the surface erosion phenomenon, it became harder to observe biodegradation, length and diameter changes after some point. Partial biodegradation for the micro-swimmers in 204 hours is consistent with the literature, where full degradation was not observed after several weeks for most of the studies.

In addition to biodegradation, in vitro biocompatibility of the degradation products was investigated using SKBR3 breast cancer cells. The SKBR3 cells were treated with the degradation products of degraded micro-swimmers 10 for one day and then stained with live-dead assay for toxicity analysis. The results showed that the degradation product of the micro-swimmers 10 did not have a toxic effect on SKBR3 cells and the live/dead cell ratio for both control and treated groups were similar and approximately 90% of the whole cell populations (FIGS. 3D and 3E).

In this connection FIG. 3D shows schematic representations of optical microscopy images of live-dead staining of SKBR3 breast cancer cells with FIG. 3Di showing non-treated and FIG. 3Dii showing cancer cells treated with the degradation products of the micro-swimmers 10 for 1 day. The hollow dots represent live cells and the full dots represent dead cells. FIG. 3E shows the results of the quantification of viability of SKBR3 breast cancer cells treated with the degradation products. The viability didn't alter in the cells treated with the degradation products (p>0.05). The error bars represent the standard deviation and this is not significant (n.s.). Thus, it is hereby shown that neither the micro-swimmers 10 nor their degradation products form toxic reactions that could lead to harm within e.g. the human body.

FIG. 4A schematically shows the reaction pathway to obtain DOX-modified micro-swimmers 10. DOX is a substance used in the treatment of liver cancers. The DOX is a cargo material 24 that can be transported by the micro-swimmers 10. In order to attach the cargo material 24, a chemical linker 26 is initially attached to a body portion 10′ of the micro-swimmer 10, e.g. by coating.

The function of the chemical linker 26 is to form a releasable link between the cargo 24 and the micro-swimmer 10 by forming a chemical bond 28 to the biocompatible micro-swimmer 10 and a chemical bond 30 to the cargo material 24 that is attachable to and transportable by the micro-swimmer 10. A further function of the chemical linker 26 is that it is capable of releasing the cargo material 24 from the micro-swimmer 10 on the application of a stimulus, for example the application of light and/or heat or in the vicinity of predefined amount of specific enzymes “due to a pathological condition”.

In this connection it should be noted that the cargo 24 respectively the cargo material 24 may be selected from the group of members consisting of enzymes, molecules, drugs, proteins, genetic materials, nanoparticles, radioactive seeds for therapeutic or diagnostic purposes and combinations of the foregoing.

In this connection it should further be noted that the chemical linker 26 may be selected form the group of members consisting of a photo cleavable linker, preferably an NHS and Alkyne modified o-nitronezyl derivative, an enzymatically cleavable linker, for example, one of the matrix metalloproteinase recognition peptide sequences, a thermally cleavable linker, i.e. a chemical linker that has a melting point above the temperature of the body and that melts if heat is locally applied in order to release the cargo 24 and/or combinations of the foregoing.

In the example of FIG. 4A, the amino groups (NH₂) present on the micro-swimmers 10 react with NHS group of o-nitrobenzyl photocleavable linker molecules 26 to form the chemical bond 28. Then, azide-modified DOX 24 reacts with alkyne ends of the micro-swimmers 10 to form the chemical bond 30.

FIG. 4B shows the DOX release from the micro-swimmers 10 exposed to an external light stimulus of 30 mW light intensity for 30 min. FIG. 4Bi shows the fluorescence intensity prior to the application of the light and FIG. 4Bii after 30 mins. The decrease in the fluorescence intensity indicates the cleavage of DOX from the micro-swimmers 10 and its release.

FIG. 4C shows the DOX release from the micro-swimmers 10 using two different laser intensities for 3 mW (round points) and 30 mW light intensity (square points). On using the 30 mW light intensity 60 to 70% of the DOX is released after exposure for 30 minutes whereas only 30 to 40% of the DOX are released after exposure for 30 minutes with 3 mW light intensity. As also indicated in FIG. 4C approximately 60% of the DOX is released within the first five minutes of the exposure with 30 mW light intensity.

Two different light intensities at 365 nm wavelength, 3 mW and 30 mW, were selected to demonstrate on-demand light-triggered drug release. For 30 mW, there was significant reduction in the fluorescence intensity after 30 min which means that DOX 24 was released from the micro-swimmers 10 as indicated in FIG. 4B. Approximately 60% of the bound DOX was released within 5 min for 30 mW light intensity (FIG. 4C).

The release rate dramatically decreased after 5 min. The incomplete release is due to low photochemical conversion observed for nitrobenzyl groups. Slow release after 5 min was probably observed due to slower diffusion of DOX molecules 24 which were cleaved-off from center of the micro-swimmers 10. Slower drug release was observed in the case of 3 mW light intensity compared to 30 mW light intensity. The cumulative drug 24 release rate decreased and converged approximately to 40% (FIG. 4C). The lower drug release could be explained as slower reaction kinetics due to the lower light intensity.

Thus, by varying the light intensity one can control the amount of DOX released and hence one can tailor the type of release on application of the stimulus in dependence on the intensity of the stimulus and the time during which the stimulus is applied.

FIG. 4D shows an example of how such smart dosing of DOX 24 from the micro-swimmers 10 may take place. The solid bars at t=0 to 1 min and t=6 to 7 min illustrate the stimulus in the form of a laser with 365 nm wavelength. During the application of the stimulus approximately 15% of DOX was released per minute from the micro-swimmers 10.

A sharp drug release from the micro-swimmers 10 was observed when light was on (30 mW light intensity) for 1 min, and afterward, there was no or slight drug release from the micro-swimmers when light was off for 5 min (FIG. 4D). Approximately 15% of the total drug was released per dose. This showed that the user can control on-demand drug release profile from the micro-swimmers 10. Also, the amount of drug 24 that is dosed can be tuned by changing either light intensity or exposure time.

Thus, photocleavage-based light-triggered delivery systems 10 are shown that can be controlled to release varying rates of different drug molecules. In these systems, drug molecules 24 are chemically bound to photocleavable linker molecules 26. The photocleavable linker molecules 26 e.g. split into two parts upon light radiation and drug molecules 24 are released from the attached structures. o-nitrobenzyl is a photocleavable group 24 and functional o-nitrobenzyl derivatives have been used for delivery of various biomolecules. o-nitrobenzyl derivative that has N-Hydroxysuccinimide ester (NHS) and alkyne can be quite effective for the release of molecules 24 due to its chemical functionality.

NHS groups selectively react with amino groups (known as NHS-Amine coupling) to form the chemical bond 28 and alkyne groups react with azide groups (known as copper (I) catalyzed Click reaction) to form the chemical bond 30. The NHS end of photocleavable linker molecules 26 were conjugated to free amino groups of the micro-swimmers 10. Then, azide-modified DOX, which was utilized as a model drug 24, is linked to the alkyne ends of the attached photocleavable linker molecules 26 forming the chemical bond 30.

Thus, two different chemical reactions were performed to obtain DOX-functionalized micro-swimmers 10 (FIG. 4A). In the first step, the micro-swimmers 10 were treated with the o-nitrobenzyl photocleavable linker molecules 26 containing solution. Alkyne-ended micro-swimmers 10 were obtained after this, so-called NHS-Amine, coupling reaction. As a second step, alkyne ended micro-swimmers 10 were treated with azide-DOX containing reaction mixture 24.

The smart dosing of therapeutics 24 is another important consideration of various delivery systems 10 since many drugs 24 have serious off-target side effects. As presented, a controlled drug release 24 from the micro-swimmers 10 is possible by on-demand switching the laser light on and off.

FIG. 5 illustrates the determination of degree of methacrylation using 2,4,6-trinitrobenzene sulfonic acid (TNBS) assay. The degree of methacrylation of ChMA macromolecules was analyzed with 2,4,6-Trinitrobenzene Sulfonic Acid (TNBS) assay which is based on the quantification of unmodified free amino groups.

Unmodified chitosan, as a control group, and 0.05% (w/v) ChMA macromolecules were respectively dissolved in 0.2% (v/v) acetic acid solution. 80 μL of the solutions were incubated with 40 μL of 2% (w/v) NaHCO₃ and 60 μL of 0.1% (v/v) TNBS reagent (Thermo Fisher Scientific) at 37° C. for 2 h.

After the incubation period, 60 μL of 1 N HCl was added into the solutions, and then absorbance of the samples was measured at 345 nm using a plate reader (BioTek Gen5 Synergy 2, Bad Friedrichshall, Germany). The degree of methacrylation was calculated according to the following equation:

$$Degree of Methacrylation \% = 100 - \frac{Absorbance of Sample}{Absorbance of Unmodified Chitosan} \times 100 \qquad ( 1 )$$

As illustrated in FIG. 5 the unreacted chitosan gives the highest absorbance at 345 nm wavelength light (in terms of amino groups). Moreover, amino groups are consumed during the reaction between chitosan and methacrylic anhydride, and this results in a gradual decrease of the absorbance at 345 nm wavelength light.

In order to be able to use the chitosan in the 3D printing process described in the foregoing the absorbance has to be lower than that for the unreacted chitosan. The chitosan reacted with 70% methacrylamide has an absorbance that is within the range required for the 3D printing process which is why this product was used.

FIG. 6 shows the microchannel setup utilized for two-photon-based 3D-printing of the micro-swimmers 10. The permanent magnets 20 are used to align SPIONs inside the prepolymer solution 14 to obtain the optimum magnetic actuation efficiency during the swimming experiments. The micro-swimmers 10 were printed longitudinally perpendicular to the magnetic field B.

FIGS. 7A & B show energy-dispersive x-ray spectroscopy elemental mappings of a respective micro-swimmer 10. The elemental mapping of the micro-swimmers 10 was performed at 15 keV for 10 min. The double helical structure can be seen in both images, the below image shows the presence of carbon atoms (hashed region) inside the micro-swimmer 10.

FIGS. 9A & B show optical microscopy images of the chemical integration of the drug molecules, i.e. of the cargo material 24, into the micro-swimmers 10. FIG. 9A shows the micro-swimmers 10 that are not treated with the chemical linker 24, i.e. those without the o-nitrobenzyl linker modification, but rather are directly treated with the drug molecules (azide-DOX).

FIG. 9B shows micro-swimmers 10 to which a chemical linker 26 is applied prior to dosing these with a cargo material 24. the o-nitrobenzyl linker modification were treated with the drug molecules 24 (azide-DOX). Images were captured with the same fluorescence intensity and exposure time, and represent the controlled integration of azide-DOX 24 onto the micro-swimmers 10.

To confirm azide-DOX 24 was bound to the micro-swimmers 10 by Click reaction, only the second step was performed with another group of micro-swimmers 10 as negative control group as indicated in FIG. 9A. In the negative control, azide-modified DOX 24 could not be bound to the micro-swimmers 10 since there was no reaction between amino and azide groups. The DOX-modified and negative control groups were compared using fluorescence microscopy.

As indicated in FIG. 9B the DOX-modified group had significantly higher and homogenous fluorescence emission in comparison to the negative group at same exposure intensity and time (FIG. 9A). Meanwhile, low fluorescence emission from the negative group was due to diffusion of the drug molecules 24 into the micro-swimmers 10. These results confirmed the chemical conjugation of o-nitrobenzyl linker and azide-modified DOX to the micro-swimmers 10.

Thus, it is generally advisable to use a chemical linker 26 in order to bond a cargo 24 to the micro-swimmers 10.

FIGS. 10A to C show photobleaching tests for the drug molecules prior to controlled release experiments. FIG. 10A shows a negative group excited with a 470 nm wavelength laser light for 30 min, with the negative group comprising micro-swimmers 10 without the chemical linker 26, i.e. without the o-nitrobenzyl linker modification. FIG. 10B shows an exposure of the negative group to a 3 mW 365 nm wavelength laser light. FIG. 10C shows an exposure of the negative group to a 30 mW 365 nm wavelength light.

Bleaching tests and controlled drug release from the micro-swimmers 10 o-nitrobenzyl linker molecules 26 between the micro-swimmers 10 and DOX 24 experienced selective bond cleavage with light irradiation at 365 nm wavelength and 3-30 mW intensity.

For drug release experiments, the main assumption was that the initial fluorescence intensity of the micro-swimmers 10 corresponds to 100% drug 24 loading to the micro-swimmers 10. The drug 24 release from the micro-swimmers 10 was characterized based on the fluorescence intensity decrease over time.

The bleaching tests were performed to confirm that there was no photobleaching- and diffusion-related fluorescence intensity changes in the micro-swimmers 10. Accordingly, the negative group was exposed to light at 365 nm, which was used for cleavage of the linker molecules 26, and to light at 470 nm, which was used for DOX 24 excitation and fluorescence intensity change analysis, wavelengths.

No decrease in the fluorescence intensity was observed for excitation both at 365 nm (FIG. 10A) and at 470 nm wavelengths (FIGS. 10B and 10C), which means that the drug molecules 24 did not lose their fluorescence upon light exposure; but the fluorescence change in the whole microsystems was due to the controlled drug 24 release.

FIGS. 11A & B show optical microscopy images that indicate a controlled and localized release of drugs 24 from the micro-swimmers 10. FIG. 11A shows a 365 nm wavelength laser light focused onto the micro-swimmers 10 found in left column (FIG. 11Ai). Drug molecules 24 were controlled released upon light exposure while the other group, found in right column (FIG. 11Aiii), still had the integrated drug molecules 24.

As indicated in FIG. 11A the drugs 24 can be released from a specific group of the micro-swimmers 10 while others retained the drug inside (FIG. 11A). Moreover, FIG. 11B shows how it is possible to release drugs 24 from half of the micro-swimmers 10 further indicating the local control one can have over the release of the cargo material 24, by varying the position of the focal point of the applied laser light (not shown).

The micro-swimmers 10 discussed in the foregoing can be used for a targeted delivery of the cargo material 24 at desired target regions, e.g. within the liver or kidney of the human or animal body (respectively not shown). If the micro-swimmers 10 are used e.g. in the gastrointestinal tract, then these can simply be ingested by swallowing and on monitoring the natural progress throughout the human body one can then actively steer the micro-swimmer 10 once it is e.g. present within the intestine or stomach, if the micro-swimmers 10 are to be used for the delivery of cargo material 24 into e.g. the liver, then the micro-swimmer 10 is injected into a region, e.g. a blood vessels, associated with the desired target region.

Once the micro-swimmer 10 is e.g. within 1 to 2 mm of the target site, e.g. the liver tumor, the micro-swimmer 10 is directed to the desired target region with a time variable magnetic field as discussed in the foregoing. Once the micro-swimmer 10 is in the desired target region, this is stimulated in order to release the cargo 24 at the desired target region.

As discussed the step of stimulating the micro-swimmer 10 in the desired target region to release the cargo 24 is carried out by applying a light stimulus at the target region.

It is further advantageous if the step of directing the micro-swimmer 10 to the desired target region is conducted in conjunction with image mapping, e.g. using an MRI, in order to track a path of the micro-swimmer 10 to the desired target region.

As illustrated in the foregoing the cargo 24, i.e. the drug, can be released in localized manner by focusing light on the micro-swimmer 10.

In summary, a magnetically-actuated biocompatible and biodegradable chitosan-based micro-swimmer 10 was developed, which has the capability of on-demand light-triggered drug release. For this purpose photosensitive methacrylamide chitosan macromolecules were synthesized, then SPIONs were embedded therein. The micro-swimmers 10 were fabricated from this 3D polymer solution using TDLW technique. Moreover, it was demonstrated that the micro-swimmers can be actuated and steered at different frequencies under a 10 mT rotating magnetic field.

In order to show that the micro-swimmers 10 can also be used in vitro, the biodegradation of the micro-swimmers 10, without generating any in vitro cytotoxic degradation products, using a natural enzyme found in the human body is also shown.

Also shown is the combination of on-demand light-triggered drug release within the synthetic micro-swimmers 10, which makes the microsystem promising for the challenges associated with the active and controlled delivery of therapeutics 24 for the treatment of various diseases.

All the materials discussed herein were purchased from Sigma-Aldrich unless otherwise specified.

In the following certain method steps conducted to produce the micro-swimmers 10 will be discussed using the words of the inventors:

Synthesis of methacrylamide chitosan Methacrylamide chitosan (ChMA) was synthesized according to previously described protocol with some modifications. Initially, 3% (w/v) low molecular weight chitosan powder was dissolved in 3% (v/v) acetic acid solution at room temperature (RT) for 24 h. Methacrylic anhydride was added to chitosan solution at 3.5:1 w/w ratio to obtain ˜70% methacrylation degree, and the reaction was performed for 3 h with vortex mixer at RT. After performing the reaction, the reaction mixture was diluted with water and dialyzed (14 kDa cut-off) against water for 4 d. The resulting mixture was lyophilized and stored at −20° C. for further use.

3D Printing of the Micro-Swimmers

ChMA (30 mg·mL⁻¹), LAP initiator (20 mg·mL⁻¹) (Tokyo Chemical Industry Co. Ltd.) and superparamagnetic iron oxide nanoparticles (5 mg·mL⁻¹) (50 nm fluidMAG-PEG/Amine from chemicell GmbH) were dissolved in 8% (v/v) acetic acid solution. The resulted prepolymer solution was dropped on a trichloro(1H,1H,2H,2H-perfluorooctyl)silane treated glass slide and printing was performed with a commercially available direct laser writing system (Photonic Professional, Nanoscribe GmbH). After the fabrication, glass slides were thoroughly washed with ddH₂O, and then the samples were kept at 4° C. for further use.

Integration of Photocleavable Linker and Drug Molecules to the Micro-Swimmers

Initially, photocleavable o-nitrobenzyl linker (1-(5-methoxy-2-nitro-4-prop-2-ynyloxyphenyl) ethyl N-succinimidyl carbonate from LifeTein LLC) was bound to surface of the micro-swimmers through NHS-Amine coupling reaction. Briefly, 500 μM of the linker was dissolved in anhydrous dimethyl sulfoxide and the micro-swimmers were treated with the linker solution for 4 h at RT. After that, for coupling azide-modified DOX (LifeTein LLC) to the alkyne ends of the linker molecules, bound to the micro-swimmers, previously described protocol was adapted with some modifications. The micro-swimmers were treated with a solution containing 50 μM azide-modified DOX, 100 μM CuSO₄, 5 mM sodium ascorbate, 500 μM tris(3-hydroxypropyltriazolylmethyl)amine for 3 h at RT. Finally, the micro-swimmers were washed several times with ddH₂O to remove unbound drug molecules and kept in dark for further use.

Bleaching Test and Controlled Drug Release from the Micro-Swimmers

Drug integrated micro-swimmers were equilibrated to RT, washed several times with ddH₂O and kept overnight in ddH₂O. Controlled drug release from the micro-swimmers upon light exposure at 365 nm was investigated using flourescence inverted microscope (DMi8, Leica Microsystems). Time-lapse fluorescent images were acquired every 10 s for a period of 30 min. Light intensity was adjusted to either 3 mW or 30 mW, and the exposure time was set to 1 s. Fluorescence intensities of the micro-swimmers were analyzed using LASX analysis toolbox (Leica Microsystems). On-demand controlled drug release experiment was performed by 1 min of light exposure followed by 5 min of refractory period. In both cases, background fluorescence was subtracted from the measured values.

Fluorescence bleaching of the micro-swimmers loaded with the drug molecules through passive diffusion was tested by exposure to light at 365 nm or 470 nm as in the controlled release experiments, and image acquisition. Bleaching test both for 3 mW and 30 mW light power at 365 nm, and light power at 470 nm were tested for 30 min, and fluorescent images were acquired every 10 sec. Similar to release experiments, fluorescent intensities of the individual micro-swimmers were measured through LASX analysis toolbox (Leica Microsystems) and background was subtracted from the measured values.

Degradation of Micro-Swimmers and Cytotoxicity Investigation of the Degradation Products

3D printed micro-swimmers were treated with different concentrations of lysozyme solution (1.5, 15 and 150 μg·mL⁻¹), prepared in 1× phosphate buffered saline, at 37° C. The length and diameter of the micro-swimmers were measured using Nikon Eclipse Ti-E inverted microscope with 20× magnification in DIC mode with increasing time intervals (3, 6, 12, 24, 48 h). Enzyme solutions were refreshed every 12 h to prevent inactivation of the enzyme. Degradation products were used to investigate biocompatibility and cytotoxicity of the micro-swimmers. Briefly, SKBR3 breast cancer cells (passage #8) were seeded into a 96-well plate as 5000 cells/well. Then, they were treated either with the degradation products of thousand 3D printed micro-swimmers or growth medium (control group) for 1 d upon ˜80% confluence was reached. Finally, the cells were stained with live-dead imaging solution (Life Technologies) for 20 min at RT, imaged using a fluorescence inverted microscope, and counted using ImageJ for quantitative analysis.

1.-26. (canceled)

27. A method of making a biocompatible micro-swimmer, the method comprising the steps of:
providing a photo cross-linkable biopolymer solution;
adding magnetic particles and a photo initiator to the photo cross-linkable biopolymer solution to form a 3D-printable solution;
applying a laser with a variable focus directed at the 3D-printable solution;
varying the focus of the laser through the 3D-printable solution to form the biocompatible micro-swimmer with a predefined shape; and
applying a chemical linker to the biocompatible micro-swimmer having the pre-defined shape.

28. The method in accordance with claim 27, wherein the chemical linker forms a link between the biocompatible micro-swimmer and a cargo that is attachable to and transportable by the micro-swimmer.

29. The method in accordance with claim 28, further comprising the step of: attaching a cargo at the biocompatible micro-swimmer via the chemical linker.

30. The method in accordance with claim 29, wherein the cargo is selected from the group of members consisting of enzymes, molecules, drugs, proteins, genetic materials, nanoparticles, radioactive seeds for therapeutic or diagnostic purposes and combinations of the foregoing.

31. The method in accordance with claim 28, wherein the chemical linker is selected such that the link between the micro-swimmer and the cargo can be released on the presence of a stimulus.

32. The method in accordance with claim 27, wherein the chemical linker is a photo cleavable linker.

33. The method in accordance with claim 27, wherein the chemical linker is one of an enzymatically cleavable linker and a thermally cleavable linker.

34. The method in accordance with claim 27, wherein the photo-cross-linkable biopolymer solution is a solution comprising bioactive, biodegradable polymers, biocompatible polymers, gelatine, alginate, polypeptides, nucleic acids, polysaccharides and combinations of the foregoing.

35. The method in accordance with claim 27, wherein the magnetic particles have a size selected in the range of 5 nm to 200 nm.

36. The method in accordance with claim 27, wherein the magnetic particles are selected from the group of members consisting of iron oxide particles, iron platinum particles, neodymium iron boron particles, aluminum nickel cobalt particles, iron particles, cobalt particles, and samarium cobalt particles.

37. The method in accordance with claim 27, wherein the photo initiator is a molecule that upon two photon absorption splits into half and generates radicals that initiates the photo-crosslinking.

38. The method in accordance with claim 27, wherein the cargo is releasable from the micro-swimmer on the application of a stimulus or in the vicinity of predefined amount of specific enzymes.

39. The method in accordance with claim 27, further comprising the step of: applying a magnetic field whose magnetic field strength is selected in order to align the magnetic particles within the 3D-printable solution during the step of applying the laser.

40. The method in accordance with claim 27, wherein the micro-swimmer has a shape that is configured to be asymmetrically moved in dependence on time in the presence of a rotating magnetic field, such as a helical or double helical shaped structure.

41. The method in accordance with claim 27, wherein the micro-swimmer has an elongate shape, with a ratio of length to width being selected in the range of 2:1 to 10:1 and/or
wherein at least one dimension of the micro-swimmer is selected in the range of 0.0001 to 1 mm.

42. A biocompatible micro-swimmer, the micro-swimmer comprising a body portion formed of a 3D printable solution including a photo cross-linkable biopolymer solution, magnetic particles and a photo initiator;
wherein the body portion of the micro-swimmer has a shape that is configured to be asymmetrically moved in dependence on time in the presence of a rotating magnetic field; and wherein the body portion is coated with a chemical linker.

43. The biocompatible micro-swimmer in accordance with claim 42, wherein the body portion of the micro-swimmer has an elongate shape, with a ratio of length to width being selected in the range of 2:1 to 10:1; and/or
wherein at least one dimension of the micro-swimmer is selected in the range of 0.0001 to 1 mm; and/or
wherein the micro-swimmer is configured to be moved with a Reynold's number of less than 0.1; and/or
wherein the micro-swimmer is magnetised in a direction perpendicular to its major axis.

44. The biocompatible micro-swimmer in accordance with claim 42, wherein at 1.5 μg/ml Lysozyme concentration a length of the micro-swimmer degrades to a length of at most 70% of the initial length and a diameter of the micro-swimmer degrades to a diameter of at most 50% of the initial diameter of the micro-swimmer within a period of time of 210 hours.

45. A method of using one biocompatible micro-swimmer, the micro-swimmer comprising a body portion formed of a 3D printable solution including a photo cross-linkable biopolymer solution, magnetic particles and a photo initiator;
wherein the body portion of the micro-swimmer has a shape that is configured to be asymmetrically moved in dependence on time in the presence of a rotating magnetic field; and wherein the body portion is coated with a chemical linker, the method comprising the steps of:
providing the micro-swimmer in a region associated with the desired target region;
directing the micro-swimmer with a time variable magnetic field to the desired target region;
stimulating the micro-swimmer in the desired target region to release the cargo.

46. The method in accordance with claim 45, wherein the step of directing comprises the application of a rotating magnetic field having a magnetic field strength in the range of 5 mT to 50 mT with a frequency selected in the range of 1 Hz to 50 Hz; and/or
wherein the step of stimulating the micro-swimmer in the desired target region to release the cargo is carried out by applying a light stimulus at the target region; and/or
wherein the step of directing the micro-swimmer is conducted in conjunction with image mapping in order to track a path of the micro-swimmer to the desired target region.
